(19) United States
(12) Patent Application Publication
(10) Pub. No.: US 2008/0039408 A1
(43) Pub. Date: Feb. 14, 2008

(54) Use of Oxypurinol as an Inhibitor of Anti-Neoplastic Agent-Induced Cardiotoxicity

(76) Inventor: Ronald Freudenberger, Princeton, NJ (US)

Correspondence Address: KENYON & KENYON LLP, ONE BROADWAY, NEW YORK, NY, 10004, US

(21) Appl. No.: 11/792,598

(22) PCT Filed: Dec. 6, 2005
(86) PCT No.: PCT/US05/44332

§ 371 Date: Oct. 29, 2007

Related U.S. Application Data
(60) Provisional application No. 60/633,840, filed on Dec. 6, 2004.

(51) Int. Cl. A61K 31/522; A61K 31/704; A61P 35/00

(52) U.S. Cl. 514/34; 514/263.3; 514/263.31

(57) ABSTRACT

The disclosure relates to methods of ameliorating or preventing cardiotoxicity caused by anti-neoplastic agents, such as doxorubicin, by administering to a patient oxypurinol, salt or derivatives thereof, before, during or after administration of the anti-neoplastic agent.

### FIELD OF THE INVENTION

The present invention relates to the novel use of oxypurinol, salts or derivatives thereof, in ameliorating or preventing adverse cardiac events including, but not limited to, those induced by the use of anti-neoplastic agents in cancer patients. More particularly, oxypurinol is used to ameliorate or prevent cardiotoxicity in patients undergoing treatment with, or in patients who have been treated with, anthracyclines, such as doxorubicin, substitutes, derivatives or combinations thereof.

### BACKGROUND OF THE INVENTION

Cancer is the second leading cause of death in the United States, exceeded only by heart disease. In 2004, the American Cancer Society estimates that 1,368,030 new cases are expected to be diagnosed. In the U.S. alone, 563,700 of the 9.6 million with a history of cancer and living with cancer are expected to die. Anti-neoplastic agents or chemotherapeutic agents that are cytotoxic to cancer cells have traditionally been the therapy of choice in treatment of cancers. However, these agents have been limited by serious severe side effects, such as the development of resistance in tumor cells or toxicity in healthy cells. About 60% of treated cancer patients suffer from such toxicities and it is estimated that 20%-30% die from complications associated with treatment as opposed to the disease itself. (Gitkin A. A. and Birchenough, J. The Cancer Matrix: Exploring the Future of Cancer Therapy. USB Warburg White Paper 2001). A common complication of anti-neoplastic toxicity is cardiotoxicity.

Of all the anti-neoplastic or chemotherapeutic agents currently being used in a clinical setting, anthracylines rank among the most effective anti-cancer drugs ever developed. (See, Weiss, R. B., The anthracycline: Will we ever find a better doxorubicin, 19 Semin. Oncol. 670-686 (1992)). Anthracyclines were first isolated from Streptomyces peucetius in the 1960s; the most important of which are and doxorubicin (DOX) and daunorubicin (DNR). Chemically, DOX is known as 14-hydroxydaunomycin and is commonly known by its trade name, Adriamycin®.

Structurally, both DOX and DNR are very similar in that they share aglyconic and sugar moieties. However, the side chain of DOX terminates with a primary alcohol whereas DNR terminates with a methyl group. This difference has important consequences in that it determines the specificity of the molecule against various cancer cell types. DOX plays an essential role in the treatment of breast and bladder carcinoma, childhood solid tumors, soft tissue sarcomas and aggressive lymphomas. DNR is active against acute lymphoblastic or myeloblastic leukemias.

An understanding of the pharmacokinetic-pharmacodynamic relationships of anthracycline activity and toxicity is of critical importance for the design of new and better treatment strategies that are more potent in their cancer-killing activity but less toxic to myocytes or other healthy cells. Despite extensive use of anthracyclines in clinical settings, the modes of action of anthracyclines still remain the subject of much controversy. (See, Minotti G. et al., Anthracyclines: Molecular Advances and Pharmacologic developments in Antitumor activity and Cardiotoxicity, 56(2) Pharmacological Review 185-229 (2004)). Briefly, mechanisms that have been considered include (1) intercalation of anthracycline molecules into DNA, which lead to the inhibition of macromolecule syntheses; (2) binding of the anthracyclines on DNA and alkylation; (3) DNA-cross linking; (4) interference with DNA unwinding or DNA strand separation and helicase activity; (5) direct effects on membranes; (6) initiation of DNA damage via inhibition of topoisomerase II; (7) induction of apoptosis in response to topoisomerase II inhibition; and (8) generation of free radicals, leading to DNA damage or lipid peroxidation.

Anthracycline-Associated Cardiotoxicity

Despite its efficacy in cancer treatment, anthracyclines are also best known to cause irreversible cardiotoxicity, even years after completion of treatment. Cardiotoxicity may occur in >20% of patients treated with doxorubicin (DOX), daunorubicin (DNR) or fluorouracil (FU), and thus, remains a significant and dose-limiting clinical problem. (See, Review, Pai, V. B and Nahata M. C., Cardiotoxicity of Chemotherapeutic Agents: Incidence, Treatment and Prevention, 22(4) Drug Safety 263-302 (2000)). Other classes of chemotherapeutic agents associated with cardiotoxicity side effects are alkylating agents, and anti-microtubule agents, such as the vinca alkaloids.

Anthracycline-associated cardiotoxicity includes cardiac events or episodes that are categorized as acute or chronic (early or late onset). Cardiac events or episodes may include mild blood pressure changes, thrombosis, electrocardiographic (ECG) changes, arrthymias, myocarditis, pericarditis, myocardial infarction (MI), cardiomyopathy, cardiac failure (left ventricular dysfunction or failure) and congestive heart failure (CHF). These may occur during or shortly after treatment, within days or weeks after treatment and may not be apparent until months or years after completion of chemotherapy.

An acute cardiac event generally occurs within a week after a single dose or a single course. Symptoms of an acute cardiac event after chemotherapeutic treatment include acute or subacute ECG changes, sinus tachycardia, arrthythmias, pericarditis, myocarditis, chest pains, hypotension, irregular pulse, agina, MI, and/or cardiogenic shock. Acute cardiotoxicites, such as arrhythmias, and transient, non-specific ECG abnormalities are usually self-limiting and are rarely life threatening. The incidence of acute or subacute ECG changes is about 20%-30% and arrhythmias is about 0.5%-3%.

In contrast, chronic anthracycline-associated cardiotoxicity, characterized by cardiomyopathy, is frequently life threatening. Chronic cardiac events can be subdivided into early onset or late onset. An early onset chronic event generally occurs within a year after completion of chemotherapy, from within 0-231 days, and appears to be dose dependent. For example, the incidence of early onset chronic cardiotoxicity is 1-2% at a dose of <400 mg/m² and increases to 7% at a dose of 550 mg/m².

A late onset chronic event generally occurs within 4-15 years after completion of chemotherapy. Late onset chronic cardiac events occur in 18-65% of patients treated with DOX. Chronic cardiac events are often characterized by congestive heart failure, and also includes palpitations, cardiac tamponade, pulmonary congestion, cardiomegaly, pericardial effusion and ECG changes. Late onset chronic anthracycline-induced cardiotoxicity also causes dysfunction of the right and/or left ventricle of the heart (see, Steinherz, L. J., et al., Cardiac toxicity 4 to 20 years after completing anthracycline therapy, 266 JAMA 1672-1677 (1991); Schwartz, R. G., et al., Congestive heart failure and left ventricular dysfunction complicating doxorubicin therapy. Seven year experience using serial radionuclide angiocardiography, 82 Am. J. Med. 1109-1118 (1987)), arrthymias, and heart failure years or decades after chemotherapy. Further, histologically, myofibrillar loss and sarcoplasmic vacuolation characterize chronic anthracycline-induced cardiac injury. (See, Billingham, M. E. and Bristow, M. R., Evaluation of anthracycline cardiotoxicity: Predictive ability and functional correlation of endomyocardial biopsy, 3 Cancer Treat Symp. 71-74 (1984)).

Several risk factors are known to increase the frequency and severity of anthracycline-induced cardiotoxicity, including but not limited to prior thoracic or mediastinal irradiation, age (<4 years or >70 years), female gender, history of hypertension, pre-existing cardiac disease, rate of drug administration and cumulative dosage.

Generally, the probability of developing impaired myocardial function is estimated to be 1% to 2% at a total cumulative dose of 300 mg/m² of doxorubicin, 3% to 5% at a dose of 400 mg/m², 5% to 8% at 450 mg/m² and 6% to 20% at 500 mg/m². The risk of developing CHF increases rapidly with increasing total cumulative doses of doxorubicin in excess of 450 mg/m². (See, Pharmacia Inc., at http://www.meds.com/leukemia/idamycin/adriamycin.html)

In addition, fatal cardiomyopathy has been estimated to occur in 2%-7% of patients after receiving a cumulative dose of 500 mg/m² DOX. (See, Swain, S.M. et al., Congestive Heart Failure in Patients Treated with Doxorubicin, 97 Cancer 2869-2879 (2003) and Lenaz, L. and Page, J., Cardiotoxicity of Adriamycin and related anthracyclines, 3 Cancer Treat. Rev. 111-120 (1976)). However, the National Cancer Institute cautioned that this figure may underestimate the full extent of the risk. (Heart Risks of Doxorubicin Higher than Previously Reported, http://www.cancer.gov/clinicaltrials/results/congestive-heart failure0503/).

The cause of anthracycline-induced cardiotoxicity is probably multifactorial and may be related to the secondary effects of one or more of the mechanisms of anthracycline action described above. For example, mechanisms that have been proposed to cause anthracycline-induced cardiotoxicity include, membrane pump dysfunction, disruption of calcium homeostasis, (See, Olson, R. D., et al., Sarcoplasmic Reticulum Calcium Release is Stimulated and Inhibited by Daunorubicin and Daunorubicinol, 169 Toxicol. Appl. Pharmacol, 168-176 (2000)), mitochondrial dysfunction (See, Miwa N., et. al, Adriamycin and Altered Membrane Function in Hearts, 67 Br. J. Exp. Pathol. 747-755, 1986)), sarcoplasmic reticulum impairment, (See, Shadle, S. E., et al., Daunorubicin Cardiotoxicity: Evidence for the Importance of the Quinone Moiety in Free-Radical Independent Mechanism, 15 Biochem. Pharmacol. 1435-1444, 2000)), effects on contractile apparatus (See, Boucek, R. J., Jr., et al., Contractile Failure in Chronic Doxorubicin-induced Cardiomyopathy, 29 J. Mol. Cell. Cardiol. 2631-2640 (1997)), and free-radical-mediated myocyte damage (See, Doroshow J. H., Effect of Anthracycline Antibiotics on Oxygen Radical Formation in Rat Heart, 43 Cancer Research 460-472 (1983)). Free radical formation from anthracycline has been considered to play a major role in cardiotoxicity in patients treated with DOX. The mechanism by which free radicals are generated from anthracycline is discussed below.

According to one mechanism, the anthracycline or quinone ring of DOX has been shown to be reduced to doxorubicin semiquinone by a number of NAD(P)H-oxidoreductases including, cytochrome P450, xanthine oxidase, NADH dehydrogenase, and endothelial nitric oxide synthase. The DOX semiquinone quickly regenerates its parent quinone by reducing oxygen to reactive oxygen species, such as superoxide anion (O₂.⁻), hydrogen peroxide (H₂O₂), and peroxynitrite (ONOO⁻). (See, Vasquez-Vivar et al., Endothelial nitric oxide synthase-dependent superoxide generation from adriamycin, 36 Biochemistry 11293-11297 (1997)). Each electron redox cycling of DOX results in the release of iron from intracellular storage. The released iron is available to interact with DOX molecules to form complexes that convert O₂.⁻ and H₂O₂ into more potent hydroxyl radicals (.OH), which are damaging to the surrounding cells. (See, Myers, C. E., The role of iron in doxorubicin-induced cardiomyopathy, 25 (Suppl. 10) Semin. Oncol. 10-14 (1998); Minotti, G. et al., Role of iron in anthracycline cardiotoxicity: new tunes of an old song? 13 FASEB J 199-212 (1999)).

The myocardium is more susceptible to free radical damage than any other tissue because it has comparatively less superoxide dismutase and catalase activities. In addition, glutathione peroxidase, its major defense against free radical damage is suppressed by DOX. These factors result in the accumulation of hydrogen peroxide in the myocardial cells, which is rich in iron. The excess uneliminated hydrogen peroxide is converted to hydroxyl radicals, which in turn causes severe lipid peroxidation and finally extensive destruction of mitochondrial membranes, endoplasmic reticulum and nucleic acid in the myocardial cells. Moreover, it has been proposed that the superhydroxide free radicals also crosslink sulfhydryl moieties of normal calcium-release channels, resulting in extensive efflux of calcium into the cytoplasm, depleting the calcium stores in the sarcoplasmic reticulum, and thus, leading to cell death.

Free Radical Generation and Congestive Heart Failure

Congestive heart failure (CHF) is thought to be, at least in part, a result of chronic oxidative stress that culminates in tissue damage from free radical generation. Sources of free radical generation include the action of NADH/NADPH oxidases found in macrophages and vascular endothelial cells, which are activated by angiotensin II (See, Zafari A. M., et al., Role of NADH/NADPH Oxidase-derived H₂O₂ in Angiotensin II-induced Vascular Hypertrophy, 32 Hypertension 488-495 (1998)).

Free radical formation also results as a byproduct of mitochondrial respiration. Oxidative stress results from an imbalance of free radical production and consumption. The latter is mediated primarily by extra- and intra-cellular superoxide dismutases (See, Siwik D. A., et al., Inhibition of Copper-zinc Superoxide Dismutase Induces Cell Growth, Hypertrophic Phenotype, and Apoptosis in Neonatal Rat Cardiac Myocytes In Vitro 85 Circ Res 147-153 (1999)) and by glutathione peroxidase reaction (See, Dhalla A. K., et al., Role of Oxidative Stress in Transition of Hypertrophy to Heart Failure, 28 J Am Coll Cardiol 506-514 (1996)).

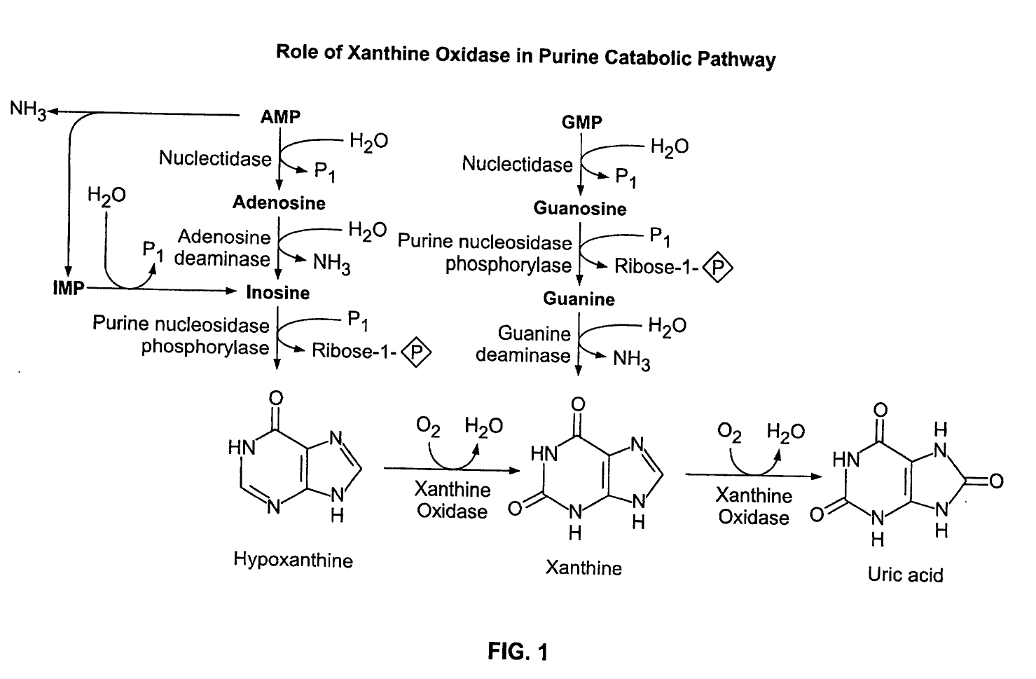

Sources of free radical generation also include the action of Xanthine oxidase which may give rise to excess free-radical generation in CHF individuals who ultimately progresses to heart failure due to impairment of myocardial function. Xanthine oxidase (XO) is a molybdenum-containing enzyme, found in the heart, that catalyzes the terminal steps of purine metabolism, converting hypoxanthine to xanthine and xanthine to uric acid (FIG. 1). (See, Hille, R. and Nishino, T., Flavoprotein Structure and Mechanism. 4. Xanthine oxidase and xanthine dehydrogenase, 11, FASEB J. 995-1003 (1995); Abadeh S., et al., Demonstration of Xanthine Oxidase in Human Heart, 20 Biochem Soc Trans 346S (1992)).

XO is believed to play a significant role in the production of superoxide and peroxynitrate in heart failure and in DOX cardiotoxicity. In addition to the formation of O₂⁻, the enzyme may also be a contributory source of nitric oxide (NO) (See, Millar T. M., et al., Xanthine Oxidoreductase Catalyses the Reduction of Nitrates and Nitrite to Nitric Oxide under Hypoxic Conditions, 427, FEBS Lett. 225-228 (1998)), especially in hypoxic conditions when nitric oxide synthase (NOS) cannot generate NO. It has been demonstrated that NO generation occurs in hypoxic rat myocardium in the presence of NOS inhibitors but not allopurinol, implicating XO in the production of NO.

Xanthine oxidase exists in two forms, XO and xanthine dehydrogenase (XDH) (See, McCord J. M., Oxygen-derived Free Radicals in Postischemic Tissue Injury, 312. N. Eng. J. Med. 159-163 (1985)). XO is a spliced variant of XDH, however, XDH may be converted to XO by either an irreversible proteolytic cleavage or reversibly by oxidation of sulfhydryl residues. Whereas XDH reduces NAD to NADH, XO utilizes molecular oxygen as an electron acceptor (See, id., and Saugstad O. D., Role of Xanthine Oxidase and its Inhibitor in Hypoxia: Reoxygenation injury, 98 Pediatrics 103-107 (1996)). Although both enzymatic forms result in O₂.⁻ formation (See, Sanders S. A., et al., NADH Oxidase Activity of Human Xanthine Oxidoreductase—Generation of Superoxide Anion, 245 Eur. J. Biochem. 541-548 (1997)), it is XO that is physiologically relevant. XDH activity is potently inhibited by NAD, which is present in abundant concentrations intracellularly (See, id., and Saugstad O. D., Role of Xanthine Oxidase and its Inhibitor in Hypoxia: Reoxygenation Injury, 98 Pediatrics 103-107 (1996)). Given the role for oxidative stress in disrupting myocardial efficiency, and the ability of allopurinol to favorably influence myocardial energetics (See, Ide T., et al., Mitochondrial Electron Transport Complex I is a Potential Source of Oxygen Free Radicals in the Failing Myocardium. 85 Circ. Res. 357-363 (1999)) and calcium sensitivity (See, Olson R. D., et al., Mechanism of Adriamycin Cardiotoxicity: Evidence for Oxidative Stress, 29 Life Sci. 1393-1401 (1981)), it is entirely reasonable to consider that this occurs via the antioxidant properties of allopurinol.

Although there is extensive experimental evidence in vitro and animal studies that support the chronic oxidative stress hypothesis, overall evidence that oxidative stress is specific to the clinical syndrome of CHF in humans is not compelling due to lack of a robust test for measuring free-radical activity in vivo in human. Thus, attempts to demonstrate a relationship between these markers and the severity of heart failure continues, particularly, with regard to the effect of the free radical cascade and its associated enzymes on cell damage. Efforts to treat CHF using free radical scavengers, such as vitamin E, vitamin C, carotene and many others show no improvement in the quality of lives in CHF individuals.

Captoril, enalapril, and other inhibitors of angiotensin-converting enzymes (ACE), beta-blockers, and aldosterone antagonists have been used to treat CHF but the results have been moderate with 30-35% reduction in mortality rates.

Xanthine Oxidase Inhibitors

U.S. Pat. No. 6,569,862 discloses the long term use of xanthine oxidase inhibitors in patients having suffered heart failure by enhancing myocardial contractility and cardiac performance through modulation of calcium sensitivity in cardiac muscle. U.S. Pat. No. 6,191,136 discloses a method of increasing calcium sensitivity of cardiac muscle by administering an effective amount of xanthine oxidase inhibitor.

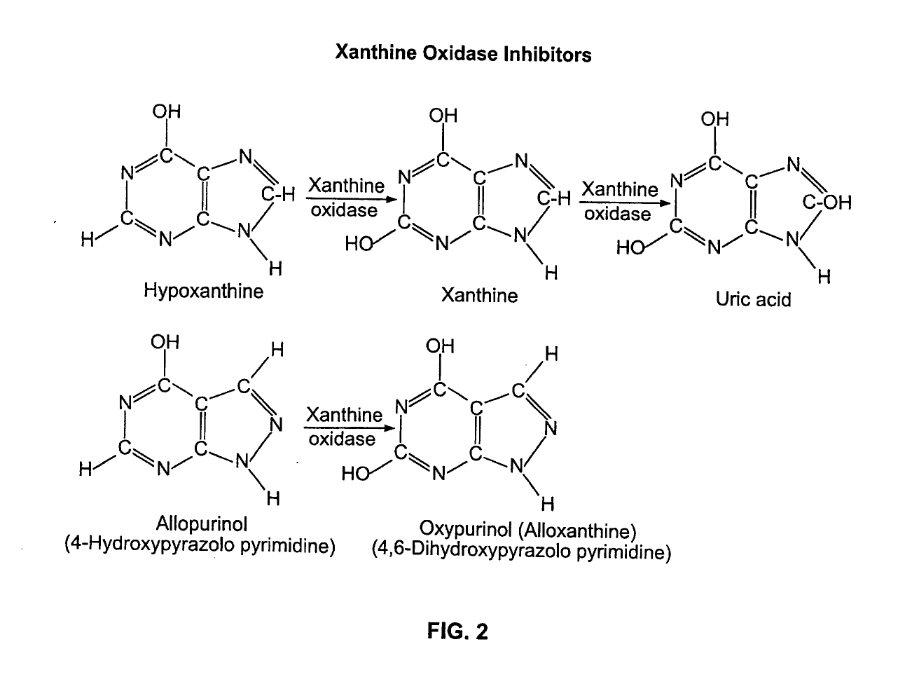

A well known xanthine oxidase inhibitor is allopurinol or 1,5-dihydro4H-pyrazolol[3,4-d}pyrimidin-4-one monosodium salt, having the structural formula set forth in FIG. 2.

Allopurinol is a structural analogue of hypoxanthine, a natural purine base. It acts on purine catabolism and is metabolized to the corresponding xanthine analogue, oxypurinol or alloxanthine (FIG. 2).

Oxypurinol is the primary metabolite of allopurinol. Both allopurinol and oxypurinol are substrates for and inhibitors of xanthine oxidase, an enzyme that converts hypoxanthine to xanthine and xanthine to uric acid, the end product of purine catabolism in man, resulting in generation of free radicals. Oxypurinol then binds to the active site of XO causing suicide inhibition. Thus allopurinol and oxypurinol cause a reduction in the production of uric acid and have, since the late 1960's, been used in treating hyperuricemia-associated with gout and some hematological disorders. (See, Physician's Drug Reference, Ed., Thompson P D R, Montvale, N.J. (2004)). A solution formulation of allopurinol is indicated for management of patients with leukemia, lymphoma and solid tumor malignancies, who are receiving cancer therapy, which causes elevation of serum and urinary uric acid levels and are intolerant to oral therapy.

Unlike allopurinol, the potency of oxypurinol is relatively unaffected by elevated concentrations of xanthine and hypoxanthine. In addition, oxypurinol produces immediate inhibition of superoxide radical production as well as progressive inhibition with time. In contrast, allopurinol, which is a substrate for XO, produces very little immediate inhibition and causes progressive inhibition only after conversion to oxypurinol. Oxypurinol is currently approved by the FDA and being used in a clinical trial for treatment of gout in allopurinol-intolerant patients.

The use of oxypurinol in the treatment of CHF is currently being investigated in three proof-of-concept studies and a Phase II-Ill clinical trial in patients with chronic heart failure. Cardiac output and exercise tolerance is used as surrogate endpoints in these studies. One of these proof-of-concept studies involves patients with CHF of ischemic aetiology and the effects of oxypurinol on left ventricular performance. Another study involves the effects of one-month of oral oxypurinol therapy on exercise capacity and the effects on left ventricular performance. (Oxipurinol: Allloxanthin, Oxyprim, Oxypurinol. 5 Drug R.D. 171-175 (2004)).

Additionally, an efficacy and safety study is being conducted of oxypurinol added to standard therapy in patients with NYHA class III-IV congestive heart failure (OPT-CHF). OPT-CHF has a double-blind, randomized parallel group, placebo-controlled design and will include patients with stable symptomatic HF in New York Heart Association (NYHA) class III-IV CHF who are deemed clinically stable on a standard and appropriately maximized heart failure therapy regimen.

Treatment of Anthracycline-Induced Cardiotoxicity

Despite its role as a double edge sword in causing cytotoxicity in tumor cells and healthy myocytes, anthracyclines such as DOX, remain a preferred chemotherapeutic agent of choice for treating solid carcinomas and soft tissue sarcomas, lymphomas, and leukemias. Since CHF is often the cause of death in anthracycline-treated patients, treatment of cardiotoxicity induced by chemotherapy in combination with cardiovascular drugs presently available may be further complicated by a possible reduction of efficacy in the activity of the chemotherapeutic agent. Thus, it remains a long felt need for drug developers to produce therapies that are not only substantially less damaging to the myocytes but at least, equally or more efficacious in preventing cancer cell growth. To date a method of treatment has not been developed that can be used prophylactically to prevent or significantly ameliorate irreversible damage to the heart muscles of patient undergoing effective chemotherapy.

Numerous attempts to identify novel anthracyclines that are superior to DOX or DNR in terms of activity and/or cardiac tolerability have been made, but only a few analogs, such as epirubicin and idarubin, have reached the stage of clinical development and approval. Although these analogs have a higher cumulative dose capacity, the risk of chronic cardiotoxicities remains.

Similarly, attempts to treat anti-neoplastic agent induced cardiomyopathy using cardiovascular drugs used in treating CHF, such as inhibitors of ACE, show only transient improvement in function and the cardiac muscles are still prone to inexorable deterioration.

In addition, it has also been proposed that use of a free radical scavenger, such as tocopherol (vitamin E) and N-acetylcysteine may assist in preventing cardiotoxicity of DOX without diminishing its anti-tumor activity. Efforts using concurrent administration of DOX and a free radical scavenger have proven to be unsuccessful.

More recently, dexrazoxane (ICRF-187) or Zinecard® (Pharmacia & UpJohn, Kalamazoo, Mich.), a cardioprotective agent, has been approved for use by FDA to reduce the incidence and severity of cardiomyopathy associated with DOX administration in women with metastatic breast cancer who have received an accumulative DOX dose of 300 mg/kg. (See, Williams, G. A., et al., FDA oncology drugs advisory committee review of Zinecard (dexrazoxane, ADR-529, ICRF-187). Rockville, Md. Center for Drug Evaluation and Research, US Food and Drug Administration 1-13 (1992)). Dexrazoxane is a bispiperazine, which is converted intracellularly to an active carboxylamine form, similar to ethylenediaminetetra-acetic acid (EDTA), and acts as a chelator of heavy metals. It, therefore, binds to intracellular iron, inhibiting conversion of superoxide anions and hydrogen peroxide to superhydroxide free radicals. (See, Thomas C, et al., The hydrolysis product of ICRF-187 promotes iron-catalyzed hydroxyl radical production via the Fenton reaction, 45 Biochem. Pharmacol. 1967-1972 (1993)). Although dexrazoxane prevents chronic cardiotoxicity in all laboratory animals tested, early results from initial double-blind, placebo-controlled trials indicate that there may be a concern over decreased efficacy of DOX when used with dexrazoxane (Pai, V. B. and Nahata, M. C., Cardiotoxicity of Chemotherapeutic Agents, 22 Drug Safety 263-302, (2000)). In addition, the impact on long-term survival is unknown. Nevertheless, Dexrazoxane is approved for use in reducing incidence and severity of cardiomyopathy associated with DOX treatment in woman with metastatic breast carcinoma who have received a cumulative dose of 300 mg/kg.

In sum, there remains a long felt need for a treatment for DOX-related cardiomyopathy, other than by costly cardiac transplantation, with a method to prevent or ameliorate anthracycline-induced cardiotoxicity without diminishing anti-tumor activity of a chemotherapeutic agent. It is envisioned that such a method will have a cardioprotective effect even with increased cumulative dose of the anthracycline administered. In addition, it is also envisioned that such a method would have its effect on an early event in the DOX-reaction cycle, either at the DNA or enzymatic level, prior to damage mediated by its metabolites and associated enzymes.

### SUMMARY OF THE INVENTION

The present invention provides methods for ameliorating or preventing adverse cardiac events in a patient who is at risk for, or who is suffering from, a neoplastic condition, by administering to the patient a biologically effective amount of an xanthine oxidase inhibitor, such as for example, oxypurinol, salts or derivatives thereof.

The present invention provides a method of ameliorating or preventing anti-neoplastic agent-induced cardiotoxicity in a patient at risk for or suffering from a neoplastic condition comprising administering:
(a) the anti-neoplastic agent, and
(b) oxypurinol, salts or derivatives thereof, in a biologically effective amount to ameliorate or prevent cardiotoxicity in said patient.

The present invention provides a method of ameliorating or preventing anti-neoplastic agent-induced cardiotoxicity comprising:
(a) identifying a patient treated with the anti-neoplastic agent; and
(b) administering oxypurinol, salts or derivatives thereof, in a biologically effective amount sufficient to ameliorate or prevent cardiotoxicity in said patient.

The present invention also provides a method of ameliorating or preventing cardiotoxicity comprising:
(a) identifying a patient suffering from a neoplastic disease at risk for or suffering from an adverse cardiac event; and
(b) administering oxypurinol, salts or derivatives thereof, in a biologically effective amount sufficient to ameliorate or prevent cardiotoxicity in said patient.
   Such methods may further comprise the step of identifying whether the patient was exposed to an anti-neoplastic agent prior to performing step (b). Such method may further comprise the step of administering an anti-neoplastic agent prior to or contemporaneous with performance of step (b).

The present invention further provides methods for ameliorating or preventing anti-neoplastic agent-induced cardiotoxicity by identifying a patient who is suffering from a neoplastic disease or at risk for, and/or suffering from an adverse cardiac event, and administering a biologically effective amount of an xanthine oxidase inhibitor, such as for example, oxypurinol, salts or derivatives thereof to the patient.

The present invention further provides methods for ameliorating or preventing anti-neoplastic agent-induced cardiotoxicity by identifying a patient treated with an anti-neoplastic agent and administering a biologically effective amount of an xanthine oxidase inhibitor, such as for example, oxypurinol, salts or derivatives thereof, sufficient to ameliorate or prevent cardiotoxicity in the patient.

The present invention further provides a method of ameliorating or preventing anti-neoplastic agent-induced cardiotoxicity by co-administering a biologically effective amount of an anti-neoplastic agent and xanthine oxidase inhibitor, such as for example, oxypurinol, salts or derivatives thereof.

The present invention further provides a method for ameliorating or preventing an anti-neoplastic agent-induced cardiotoxicity by staged administration of a biologically effective amount of an xanthine oxidase inhibitor, such as for example, oxypurinol, salts or derivatives thereof before, during, or after delivery of the anti-neoplastic agent.

In a specific non-limiting embodiment of the invention, the anti-neoplastic agent-induced cardiotoxicity is selected from the group consisting of cardiac failure, left ventricular failure, cardiomyopathy, myocarditis, pericarditis, myocardial infarction, arrthymia, blood pressure change, congestive heart failure, thrombosis, and electrocardiographic change.

In yet another specific non-limiting embodiment, the neoplastic condition is selected from a group consisting of breast carcinoma, childhood solid tumor, soft tissue sarcoma, aggressive sarcoma, acute lymphoblastic leukemia, and myeloblastic leukemia.

In an embodiment of the invention, the anti-neoplastic agent is an anthracycline, including but not limited to, doxorubicin, daunorubicin, idarubicin, detorubicin, carminomycin, epirubicin, morpholinodoxorubicin, morpholinodaunorubicin, methoxymorpholinyldoxorubicin, substitutes, derivatives, and one or more combinations thereof

In an embodiment of the invention, the anti-neoplastic agent and the oxypurinol are delivered intravenously, orally, intramuscularly, or intraperitoneally. In yet another embodiment of the invention, the oxypurinol is delivered utilizing the same method of delivery as the anti-neoplastic agent.

Furthermore, the present invention also provides for a kit for delivery of one or more doses of an anti-neoplastic agent, and one or more doses of oxypurinol, salts or derivatives thereof.

It is believed that oxypurinol is an effective xanthine oxidase inhibitor and, in the presence of one or more anti-neoplastic agents, such as an anthracycline (DOX), may be effective to ameliorate or prevent anthracycline-induced cardiotoxicity. Although not wanting to be limited to any one particular theory, applicant believes that oxypurinol reduces the formation of superoxide anion, hydrogen peroxide, and peroxynitrates by reduction of free radicals, while, at the same time not interfering with the effectiveness of an anti-neoplastic agent.

The present invention provides methods for controlling and/or inhibiting superhydroxyl radical mediated damage to the heart prior to, during, or after chemotherapy, specifically in myocytes or cardiac cells, by inhibiting free radical cascade formation, or by inhibiting the suppression of sarcomeric muscle genes, such α-actin, MLC-2 and cardiac troponin I mRNA syntheses and a non-sarcomeric muscle specific mRNA synthesis.

### DETAILED DESCRIPTION OF THE FIGURES

FIG. 1. Role of Xanthine Oxidase in Purine Catabolic Pathway.

FIG. 2. Structural formula of the xanthine oxidase inhibitors, including allopurinol and oxypurinol.

### DETAILED DESCRIPTION OF THE INVENTION

The present invention is based, in part, on the ability to reduce superoxide anion (O₂.⁻) and peroxynitrite (ONOO⁻) formation through inhibition of xanthine oxidase activity. The reduction in these free radicals is believed to lead to reduced cardiotoxicity associated with use of anti-neoplastic agents. While not wishing to be limited to any one particular theory, applicant proposes that reduction of XO activity reduces the cytotoxic effect of increased O₂.⁻ and ONOO⁻ caused by anti-neoplastic agents such as DOX.

In order that the invention herein described may be fully understood, the following definitions are provided.

Oxypurinol is a xanthine oxidase inhibitor and includes, but is not limited to, salts or derivatives thereof. The salts of oxypurinol include, but are not limited to, sodium, potassium, calcium and other metal salts. Derivatives of oxypurinol include, but are not limited to, conjugates with a radionuclide, an anti-neoplastic agent, a small molecule, and or an antibody.

An “anti-neoplastic agent”, “anti-cancer agent” or “chemotherapeautic agent” is an agent with anti-cancer activity that inhibits or halts the growth of cancerous cells or immature pre-cancerous cells, kills cancerous cells or immature pre-cancerous cells, increases the susceptibility of cancerous or pre-cancerous cells to other anti-neoplastic agents, and/or inhibits metastasis of cancerous cells. These agents may include chemical agents as well as biologic agents.

Exemplary anti-neoplastic agents include, but are not limited to, anthracyclines, such as doxorubicin (adriamycin), daunorubicin, idarubicin, detorubicin, carminomycin, epidubicin, esorubicin, morpholinodoxorubicin, morpholinodaunorubicin, methoxymorpholinyldoxorubicin, methoxymorpholinodaunorubicin and methoxymorpholinyldoxorucicin, mitoxantrone and any and all substituted derivatives, combinations and modifications thereof. Further, exemplary anti-neoplastic agents include alkylating agents, such as cyclophosphamide, ifosfamide, cisplatinmitomycin, carmustine and substituted derivatives, combinations and modifications thereof. Still further exemplary anti-neoplastic agents include anti-metabolites, such as fluorouracil and cytarabine. Further examples of anti-neoplastic agents are anti-microtubule agents, such as paclitaxel and vinca alkaloids class agents, including but not limited to, vindesine, vinblastine and vinorelbine, and etoposide. Further examples of anti-neoplastic agents, include but not limited to, antibodies and fragments thereof, such as HERCEPTIN® (Trastuzumab) which recognizes and bind to surface receptor, HER2.

A “neoplastic condition” is the abnormal growth or uncontrolled proliferation of cells that is characterized by a partial or complete lack of structural organization and functional co-ordination with normal cells. A neoplastic condition includes a solid or a non-solid tumor. Solid tumors include, but are not limited to, human soft tissue sarcomas and solid carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic carcinoma, breast carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical carcinoma, testicular carcinoma, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma and childhood solid tumor.

Examples of non-solid tumors include, but are not limited to, leukemias, multiple myelomas and lymphomas. Examples of leukemias include, but are not limited to acute myelocytic leukemia (AML), chronic myelocytic leukemia (CML), acute lymphocytic leukemia (ALL), chronic lymphocytic leukemia (CLL), erythrocytic leukemia or monocytic leukemia. Examples of lymphomas include, but are not limited to, lymphomas associated with Hodgkin's disease and Non-Hodgkin's disease.

A “cardioprotective agent” is an agent that protects the cardiac muscles or myocytic cells from damage. Preferably, the cardioprotective agent protects the cardiac muscles or myocytes from damage resulting from oxidative stress or free radicals production during chemotherapy. More preferably, the cardioprotective agent protects the cardiac muscles or myocytes by inhibiting the formation of free radicals.

“Cardiotoxicity” means having a toxic effect on the heart, which includes cardiac events, such as but not limited to, mild blood pressure changes, thrombosis, electrocardiographic (ECG) changes, arrthymias, myocarditis, pericarditis, myocardial infarction (MI), cardiomyopathy, cardiac failure (left ventricular dysfunction or failure) and congestive heart failure (CHF).

“Free radicals” are atoms or molecules with an unpaired electron. They are highly reactive oxygen by-products of normal cell metabolism, which often cause cellular damage. Examples of free radicals include superoxide anions, hydrogen peroxides, hydroxyl radicals and peroxynitrite which can form by enzymatic reaction in the cells of cardiac muscles and myocytes. These enzymes include NAD(P)H-oxidoreductases, such as but not limited to, cytochrome P450, xanthine oxidase, NADH dehydrogenase and endothelial nitric oxide synthase. Therefore, inhibitors of these enzymes may result in decreased formation of free radicals and hence prevent damage to cells by the action of free radicals.

A “biologically effective amount” refers to an amount of composition sufficient to affect biological function of an organism, or part thereof. As used herein a “biologically effective amount” refers to an amount of composition sufficient to modulate the formation of free radicals and thereby modulate the level of cardiotoxicity.

“Method of delivery” refers to the manner by which a composition is administered to a patient. Methods of delivery include, but are not limited to, intravenous (IV), oral, intramuscular, subcutaneous, intratracheal, intraperitoneal, intralymphatic, nasal, transdermal or interleural, or a combination of one or more delivery method.

The present invention provides methods for ameliorating or preventing adverse cardiac events that are life-threatening in a patient who is at risk for, or who is suffering from, a neoplastic condition, a biologically effective amount of oxypurinol, salts or derivatives thereof. As used herein, “prevent” does not require 100% prevention and includes, for example, delay in onset of disease or condition.

In a non-limiting embodiment of the invention, adverse cardiac events include cardiac failure, left ventricular failure, cardiomyopathy, myocarditis, pericarditis, myocardial infarction, arrthymia, blood pressure change, congestive heart failure, thrombosis and electrocardiographic change. A preferred embodiment of the invention is congestive heart failure.

In another non-limiting embodiment of the invention, the neoplastic condition is selected from the group consisting of breast carcinoma, solid or soft tissue tumor, lymphoma and leukemia. Preferably, the neoplastic disease is lymphoma.

The present invention further provides a method for ameliorating or preventing cardiotoxicity by identifying a patient who is suffering from a neoplastic disease, and/or at risk for an adverse cardiac event and administering a biologically effective amount of oxypurinol, salts or derivatives thereof, to the patient.

The present invention also provides methods for ameliorating or preventing anti-neoplastic agent-induced cardiotoxicity by identifying a patient treated with the anti-neoplastic agent and administering a biologically effective amount of oxypurinol, salts or derivatives thereof, sufficient to ameliorate or prevent cardiotoxicity in the patient.

Such anti-neoplastic agents include, but are not limited to, a cytotoxic agent or a chemotherapeutic agent, preferably an alkylating agent, an anti-microtubule agent, an anti-metabolite, substituted derivatives, combinations and modifications thereof. Preferably, the anti-neoplastic agent is an anthracycline. A preferred anthracycline is doxorubicin (DOX), which includes substituted derivatives, combinations and modifications thereof.

In a non-limiting embodiment of the invention, the patient is screened for prior treatment of an anti-neoplastic agent. In another non-limiting embodiment of the invention, the patient is screened for prior exposure to an anthracycline, preferably DOX.

In another non-limiting embodiment of the invention, the person screened for past treatment by anti-neoplastic agent is administered a total cumulative dose of 400 to 1,000 mg/m², preferably 500-900 mg/m² and most preferably 550 mg/m² DOX. In another aspect of the embodiment, the total cumulative dose is administered over a single course of a 21-day cycle. Typically a patient is given dosages of 50-75 mg/m² per course over 4-5 courses.

In another non-limiting embodiment of the invention, the identified patient has a cardiovascular disorder, including but not limited to, an individual with a genetic predisposition to heart diseases such as cardiac failure, left ventricular failure, cardiomyopathy, myocarditis, pericarditis, myocardial infarction, arrthymia, blood pressure change, congestive heart failure, thrombosis and elecrocardiographic change.

In another non-limiting embodiment of the invention, the identified patient has a history of treatment by mediastinal or thoracic radiation.

The present invention further provides a method of ameliorating or preventing an anti-neoplastic agent-induced cardiotoxicity by staged administration to a patient an anti-neoplastic agent and a biologically effective amount of oxypurinol, salts and derivatives thereof. The staged administration, in accordance with the invention, comprises delivery to the patient of oxypurinol, salts or derivatives thereof, prior to, during, or after delivery of the anti-neoplastic agent.

In another non-limiting embodiment of the invention, oxypurinol, salts or derivatives thereof, are co-administered to the patient in combination with an anti-neoplastic agent.

In another non-limiting embodiment of the invention, the oxypurinol, salt or derivatives thereof, are administered substantially after administration of the anti-neoplastic agent.

In another non-limiting embodiment of the present invention, the oxypurinol, salt or derivatives thereof, are delivered minutes, hours, days, weeks, months or years after delivery of the anti-neoplastic agent.

In another non-limiting embodiment of the present invention, oxypurinol is administered concurrently with the anti-neoplastic agent, and by the same or different method of delivery. By concurrent, it is understood that the cardioprotective agent is separately administered during overlapping periods of administration of the anti-neoplastic agent by the same or different method of delivery. In another non-limiting embodiment of the invention, an admixture is administered containing oxypurinol together with the anti-neoplastic agent in the same method of delivery.

In another non-limiting embodiment of the invention, oxypurinol is administered sequentially with the anti-neoplastic agent by treating the patient with anti-neoplastic agent and oxypurinol, salts or derivatives thereof, in series, such that there is some or no overlap in periods of time of administration of oxypurinol and the anti-neoplastic agent. Preferably, oxypurinol is administered orally in a biologically effective amount of 600 mg/day to 1,000 mg/day. Preferable DOX dosage include, amount of 1 to 1,000 mg/m².

In a non-limiting embodiment of the present invention, any suitable methods of administration of oxypurinol and anti-neoplastic agent may be used, including but not limited to intravenous, oral, intramuscular, subcutaneous, intratracheal, intraperitoneal, intralymphatic, nasal, transdermal or intrapleural, or a combination of one or more such methods of delivery that is the same or different from that used in delivery of the oxypurinol and anti-neoplastic agent. For example, both the anti-neoplastic agent and oxypurinol may be delivered intravenously at the same or at different times, or the anti-neoplastic agent can be delivered intravenously while the cardioprotective agent can be delivered orally.

For intravenous administration, the formulation preferably will be prepared so that the amount administered to the patient will be a biologically effective amount. Doses include approximately 200-600 mg/day of oxypurinol and a cumulative dose of 125-660 mg/m² of DOX. The dosage of the present invention is effective over a wide range and depends on factors such as the pharmaceutical composition, mode of administration of the pharmaceutical, particulars of the patient to be treated or diagnosed, as well as other parameters deemed important by the attending physician.

For oral administration, the method of administration may be in any suitable form, including but not limited to tablets, liquids, emulsions, suspensions, syrups, pills, caplets and capsules. Methods for making pharmaceutical compositions are well known in the art. (See, e.g. Remington, The Science and Practice of Pharmacy, Alfonso R. Gennaro (Ed.) Lippincott, Williams & Wilkins (Pub)).

Pharmaceutical compositions may comprise conventional pharmaceutically acceptable diluents, excipients, carriers and fillers. Tablets, pills, caplets and capsules may include conventional excipients such as lactose, starch and magnesium stearate. Conventional enteric coatings may be used. Injectable solutions comprise sterile pyrogen-free media such as saline, and may include buffering agents, stabilizing agents or preservative.

The pharmaceutical composition of the present invention may also include a biologically effective amount of iron chelator, such as, but not limited to dexrazoxane.

The method in the instant invention may be used as an adjuvant therapy, after primary treatment of the tumor has been completed. Primary treatment includes resection or removal of the tumor mass followed by adjuvant therapy such as chemotherapy in combination with a cardioprotective agent.

The present invention further provides for a kit for delivery of one or more doses of an anti-neoplastic agent and one or more doses of oxypurinol, salt or derivatives thereof. In a non-limiting embodiment of the invention, the kit comprises the combination of an IV containing a dose of anti-neoplastic agent and an IV containing a dose of oxypurinol, salts or derivatives thereof.

### EXAMPLES

The following examples 1-6 are set forth to aid in understanding the invention, but are not intended and should not be construed, to limit its scope in any way. Although specific reagents and conditions are described, modifications can be made that are meant to be encompassed by the scope of the invention. The following examples, therefore, are provided to further illustrate the invention. Examples 1-5 prophetically illustrate the expected positive results of clinical studies of using oxypurinol to ameliorate or prevent cardiotoxicity caused by the administration of anti-neoplastic agents to a patient, which are further supported by actual positive results discussed in example 6.

### Example 1

### A Method for Ameliorating or Preventing Anti-Neoplastic Agent-Induced Cardiotoxicity in a Patient Undergoing Chemotherapy

Patient Selection Criteria

All patients to be considered for inclusion treatment must be receiving anti-neoplastic agent DOX for either first or recurrent treatment of any malignancy, and willing to undergo cardiac biopsy for determination of anthracycline toxicity.

In addition, patients with histologically proven, advanced metastatic carcinoma of the breast will also be considered. To be eligible, a patient must have recovered from side effects resulting from previous therapies. Patients who have been previously treated with DOX or other anthracyclines are not considered for this particular study. Patients who had other prior adjuvant chemotherapy can be considered if a recurrence is found within six months after completion of therapy. Patients who have a prior history of other anti-neoplastic hormonal therapy such as tamoxifen citrate, megesterol acetate and other hormonal therapies can also be considered for participation if therapy is withdrawn for at least two weeks or if the tumor has rapidly progressed during hormonal treatment.

Patients with prior radiotherapy will also be eligible. Criteria for eligibility includes (1) completion of radiation at least two weeks prior to commencement of study, (2) less than 50% of the pelvic bone structure is irradiated and (3) detection of measurable disease outside the field of irradiation.

In addition to the above, the patient must have an adequate performance status based on a scale of 0-4 defined by the Eastern Cooperative Oncology Group (ECOG). An ECOG of 0 indicates full ambulatory performance and patient is asymptotic while an ECOG of 4 indicates a bed-ridden patient who is incapable of normal activity. Only patients with ECOGs of 0-3 are considered. Other considerations for inclusion in the study includes adequate bone marrow function (white blood cell count greater than 4,000/mm³, platelet count of more than 100,000/mm³); renal function of less than 2 mg/dl to about 5.0 mg/dl of creatinine and hepatic function of less than 3 mg/dl bilirubin. Patients with metastatic disease in the liver are excluded.

Patients are also excluded from the study if they have a history of CHF, history of significant obstructive valvular heart disease, obstructive hypertrophic cardiomyopathy or active myocarditis, unstable angina symptoms, MI, stroke or cardiac surgery (including percutaneous intervention) within three months prior to baseline, known hypersensitivity to xanthine oxidase inhibitors, serum creatnine greater then 5.0 mg/dl, have received another investigational drug or device within 30 days prior to screening, current substance abuse, symptomatic hyperuricemia which currently requires treatment with allopurinol and which cannot be effectively treated with uricosurics or cholchicine. In addition, patients will be excluded if they are unable to have LVEF monitored, female and pregnant, nursing or of childbearing potential and not practicing effective contraceptive methods, considered high risk for non-compliance and/or deemed to be at risk for tumor lysis syndrome as determined by the primary treating oncologist will be excluded.

Informed consents are obtained from patients included in the study.

The study design following screening is carried out as set forth in TABLE I.

TABLE I
Study Flow Chart

| | | Treatment & Follow-up | | | |
|---|---|---|---|---|---|
| | | Baseline/ | | | |
| | Screening | Randomization⁸ | | | |
| Visit No. | −1 | Visit 0 | Telephone | Visits | Final Visit |
| | | | Follow-up | 1-5 | 6 |
| Day/wk/. No. | −2 Wk. | Day 0 | Days 7-10 | Wk. 4, 8, | 24 weeks |
| | | | | 12, 16 & 20 | |
| Informed consent | X | | | | |
| Incl./Excl. criteria | X | X | | | |
| Medical history¹ | X | | | | |
| HF Hosp/ER Hx | X | | | | |
| Complete physical exam | X | | | | X |
| Review qualifying LVEF | X | | | | |
| Urine pregnancy⁴ | | X | | | |
| NYHA class | X | X | | X | X |
| Concomitant medications | X | X | | X | X |
| Adjust background therapy | | | | X | X |
| Interim history | | X | | X | X |
| Brief physical exam | | X | | X | |
| Confirm stability⁵ | | X | | | |
| Up-titrate⁷ | | | X | | |
| ECG | | X | | | X |
| Clinical chemistry^{2 and 3} | X | | | X² | X |
| Hematology² | X | | | X² | X |
| Urinalysis² | X | | | X² | X |
| c-MRI⁶ | | X | | | X |
| Patient Global Heart Failure | | | | X | X |
| Assessment | | | | | |
| QOL | X | X | | X | X |
| Randomize & start study | | X | | | |
| medication | | | | | |
| Dispense study medication | | X | | X | X¹¹ |
| 6 minute walk (every 4 weeks) | X | X | | X | X |
| Schedule next visit | X | X | | X | X¹¹ |
| Document AEs/Endpoints | | X | X | X | X |

¹Include etiology & duration of HF, demographics.
²Clinical Chemistry and Hematology and Urinalysis is performed at screening, and at weeks 4, 12 and 24
³Includes Complete chemistry panel with uric acid evaluation (Serum Uric Acid results will be blinded)
⁴Performed on all women <55 years old of childbearing potential
⁵Includes adverse events, hospitalizations, emergency room/or emergent clinic/office visits since last visit.
⁶Obtain in all patients for baseline and follow-up LVEF. Baseline results are not used for “qualifying”.
⁷May adjust study medication and background therapy anytime as needed after randomization
⁸Patient may return for repeat baseline once before randomization if necessary
⁹At the 4-week visit a blood sample for population PK analysis will be obtained.
¹⁰VO2 Max Tests are to be performed following randomization at only weeks 4 and 12 and 24.
¹¹Complete for all patients entering the follow on study.

Patients selected for the study are randomized to receive 600 mg oxypurinol daily or identical appearing placebo (6 capsules). Different doses of oxypurinol are administered based on serum creatinine levels in the patient (see TABLE II).

TABLE II
Dosage Guidance base on Serum Creatinine Levels

| Serum Creatinine mg/dL | Allowable Dose |
|---|---|
| ≦2.0 | 600 mg/day |
| | 6 Capsules/Day |
| | Active/Placebo |
| >2.0 but <3.0 | 300 mg/day |
| | 3 Capsules/Day |
| | Active/Placebo |
| ≧3.0 but ≦3.5 | 200 mg/day |
| | 2 Capsules/Day |
| | Active/Placebo |
| >3.5 but <5.0 | 100 mg/day |
| | 1 Capsules/Day |
| | Active/Placebo |
| ≧5.0 | 0 mg/day |
| | 0 Capsules/Day |
| | Active/Placebo |

Oral Administration of Oxypurinol

The dose of DOX to be administered will be determined by the treating oncologists using the standard of care for the particular tumor type and the stage of disease being treated. DOX regimen includes administration of DOX intravenously by slow IV push. Patients randomized to receive DOX+oxypurinol are given identical regimen described above plus 600 mg/day oxypurinol orally or identical appearing placebo (6 capsules) concurrently with administration of anthracyclines. Patients with reduced renal function at baseline (serum creatinine above 3 mg/dL but less than 3.5 mg/dL) will receive only 3 capsules of study medication daily throughout the trial. Patients, who experience a deterioration of renal function during the study, must have their dosage of study medication reduced to 3 capsules per day, until such time as the renal function improves and serum creatinine returns to below 3 mg/dL.

The anthracycline therapy cycle is repeated every 21 days, subject to tolerance of the patients while patients continue to receive oxypurinol as determined by the treating oncologists.

Evaluation

Patients' responses to treatment with oxypurinol and DOX toxicity are assessed functionally, and histologically. Quality of life is also assessed. Cardiac MRI (cMRI) is used to determine structure and function of the heart. Histological assessment is determined using cardiac biopsy procedures. In addition, functional determinants, including serum markers of oxidative stress are measured. These markers will allow functional determination of the effects of these agents on inhibition of XO and the measures of oxidative stress.

Cardiac Magnetic Resonance Imaging

Cardiac function is evaluated via cardiac magnetic resonance imaging (cMRI) using a 1.5 T magnet (Phillips CVMR system, Phillips Medical Systems, Netherland, B.V.), equipped with Master gradients of 30 mT/m and a slow rate of 150 T/m/s), and a five channel synergy cardiac coil.

Parallel imaging of the entire ventricle for quantitation of mass and volumes is carried out. In addition left ventricular volumes, mass and ejection fraction (LVEF) are also measured. Signal intensity on the entire myocardial and skeletal muscle slices as well as qualitative assessments of focal enhancement are carried out using the American Society of Echocardiography 17-segment model.

Histologic Assessment

Endomyocardial Biopsy: Patients are subjected to routine surveillance cardiac biopsies on a clinically indicated schedule. Endomyocardial biopsy is performed by placing an intravascular sheath upon venous access or via the right subclavian or femoral vein. A bioptome is directed under fluoroscopic guidance to the right ventricular side of the interventricular septum. At least four good biopsy specimens are obtained for each pathologic evaluation.

Endomyocardium obtained at each routine biopsy is examined histologically for morphologic changes of anthracycline toxicity using a histologic scoring system as described by Billingham et al. Table III. (See, Mason J. W. et al., Invasive and Noninvasive Methods of Assessing Adriamycin Cardiotoxic Effects in Man: Superiority of Histopathologic Assessment using Endomyocardial Biopsy. 62, Cancer Treat Rep 857-864 (1978)). Characteristic histologic changes of anthracycline toxicity include cytoplasmic vacuolization, abnormal swollen nuclei, myofibrillar lysis and swelling of the cytoplasmic reticulum. (See, Billingham, M. E., et al., Anthracycline Cardiomyopathy Monitored by Morphologic Changes. 62, Cancer Treat Rep. 865-872 (1978)).

TABLE III
HISTOPATHOLOGIC SCALE OF DOXORUBICIN
CARDIOMYOPATHY*

| Grade 0 | Within normal limits |
|---|---|
| Grade 1 | Minimal numbers of cells (<5% of |
| | total number of cells per block) |
| | with early change (early |
| | myofibrillar loss and/or distended |
| | sarcoplasmic reticulum) |
| Grade 1.5 | Small group of cells involved (5 to |
| | 15% of total number), some of |
| | which have definite change (marked |
| | myofibrillar loss and/or cytoplasmic |
| | vacuolization) |
| Grade 2 | Groups of cells (16 to 25% of total |
| | number), some of which have |
| | definite change (marked |
| | myofibrillar loss and/or cytoplasmic |
| | vacuolization) |
| Grade 2.5 | Groups of cells involved (26 to |
| | 35%), some of which have definite |
| | change (marked myofibrillar loss |
| | and/or cytoplasmic vacuolization) |
| Grade 3 | Diffuse cell damage (>35% of total |
| | number of cells) with marked |
| | change (total loss of contractile |
| | elements, loss of organelles, |
| | mitochondrial, and nuclear |
| | degeneration) |

*Adapted from Billingham M E, Mason J W, Bristow M R, Daniels J R: Anthracycline cardiomyopathy monitored by morphologic changes. Cancer Treat Rep 62: 865, 1978.

Functional Assessment

Quality of life (QOL) questionnaire data are collected from patients at baseline and at set intervals during follow-up period. The QOL questionnaire, which is based on the Minnesota Living with Heart Failure Questionnaire, consists of validated instruments to measure disease-specific health-related quality of life and depression. The survey contains 21 questions regarding patients' perception of the effects of heart failure on their daily lives and each question is rated on a scale of 0 to 5. The higher the score, the worse the quality of life. (See, Rector T S, Kubo S H, Cohn J N. Heart Fail. 1987; 3:198-209).

In addition, patients are required to complete the KCCQ, a 24-item instrument or questionnaire on physical limitation, symptoms, symptom stability, social limitation, self-efficacy, quality of life, functional status and clinical summary. Another questionnaire, a 21-item Beck Depression Inventory II (BDI) is administered at baseline and every 3 months for the first year. (See, Manual for Beck Depression Inventory II, Beck et al., The Psychological Corporation, San Antonia, Tex. (1996)).

Measurement of Utilities

The ability of patients with cardiac disease to tolerate physical activity is evaluated using the six-minute-walk test according to the recommendations of Guyatt et al. (Can. Med. Assoc J., 1985, 132:919-23) and Lipkin et al. (Br. Med J. 292:653-5).

Assessment of Oxidative Stress

Xanthine Oxidase (XO) activity: XO activity is analyzed using a modified procedure of Xia and Zweier (J. Biol. Chem, 1995, 270:18797-803) and Ekelund (Circ Res, 1999, 83:437-45) as follows. Frozen plasma and PBMC are resuspended in potassium phosphate buffer, pH. 7.8, containing 1 mmol/L phenylmethylsulfonylfluoride (PMSF) and 10 mmol/L DTT to prevent in-vitro conversion of XDH to XO. Cells are lysed and the lipid layer is removed by centrifugation at 600×g for 20 min., followed by a second centrifugation at 105,000×g for 60 min. at 4° C. The resulting supernatant is chromatographed using a Sephadex G-25 column (Amersham Bioscience, formerly Amersham-Pharmacia Biotech, Inc., Piscataway, N.J.) equilibrated with phosphate buffer. XO activity is assayed by mixing 0.1 mL of the effluent in 50 mmol/L phosphate buffer containing PMSF and DTT and 0.15 mmol/L xanthine in a 1 -mL cuvette at room temperature. The amount of uric acid produced in the reaction mixture is measured spectrometrically using a Beckman DU640 spectrophotometer at 295 nm.

NO metabolites: Nitrate and nitrite production in plasma is assayed using a chemiluminescence analyzer according to the method described in Demoncheaux et al., (Analyst. 2003 128:1281-5).

Malondialdehyde-like Activity (MDA): MDA is a low molecular weight fragment resulting from oxidant damage of polyunsaturated fatty acids, which serves as a marker of lipid peroxidation, and is readily assayed using the thiobarbituric acid reactive substances (TBARS). Plasma is incubated with phosphoric acid and thiobarbituric acid, followed by separation using a chromatographic column. The eluent is subjected to fluorescent detection at 525/560 nm. Signals are calibrated using aqueous MDA standards.

Isoprostane: Isoprostanes, also known as 8-epi-prostaglandin F2α(8-EPI), are prostaglandin-like compounds that are produced upon peroxidation of lipoproteins. Oxidative stress is assessed by measuring the concentration of isoprostane present in the plasma using an enzyme immunoassay kit (Oxford Biomedical Research, Inc., Oxford Mich.).

Intravenous Administration of Oxypurinol Before Treatment with DOX

Criteria for patient selection and randomization of patients in this study are carried out essentially as that described above.

The dose of DOX to be administered will be determined by the treating oncologists using the standard of care for that particular tumor type and the stage of the disease being treated. DOX regimen includes administration intravenously by slow IV push. Patients randomized to receive DOX+oxypurinol are given identical regimen described above plus 600 mg/day oxypurinol orally or identical appearing placebo (6 capsules) beginning one day prior to administration of anthracyclines. Patients with reduced renal function at baseline (serum creatinine above 3mg/dL but less than 3.5 mg/dL) will receive only 3 capsules of study medication daily throughout the trial. Patients, who experience a deterioration of renal function during the study, must have their dosage of study medication reduced to 3 capsules per day, until such time as the renal function improves and serum creatinine returns to below 3 mg/dL.

The anthracycline therapy cycle is repeated every 21 days, subject to tolerance of the patients while patients continue to receive oxypurinol.

Evaluation

Patients are monitored as described above.

Intravenous Administration of Oxypurinol After Treatment with DOX

Criteria for patient selection and randomization of patients in this study are carried out essentially as that described above.

For intravenous administration, the formulation preferably will be prepared so that the amount administered to the patient will be a biologically effective amount. Doses include approximately 200-600 mg/day of oxypurinol and a cumulative dose of 125-660 mg/m² of DOX. The dosage of the present invention is effective over a wide range and depends on factors such as the pharmaceutical composition, mode of administration of the pharmaceutical, particulars of the patient to be treated or diagnosed, as well as other parameters deemed important by the attending physician.

As a single agent, 60 to 75 mg/m² IV as a single dose every 21 days; or 30 mg/m² IV for 3 days every four weeks is administered. When used in combination therapy, 40 to 50 mg/m² as a single IV injection is administered every 21 to 28 days. The dose is adjusted if the patient has inadequate bone marrow reserve caused by old age, prior therapy, or neoplastic marrow infiltration. Dose modifications are recommended for the following bilirubin levels: 1.0 to 2.3 mg, reduce dose by 50%; for 3.1 to 5.0 mg. Reduce dose by 75%. Alternatively, the doses of DOX may be modified as determined by the treating oncologist based on the particular tumor type and stage of disease.

The anthracycline therapy cycle is repeated every 21 days, subject to tolerance of the patients while patients continue to receive oxypurinol.

Evaluation

Patients are monitored as described above.

### Example 2

### A Method for Ameliorating or Preventing Anti-Neoplastic Agent Induced Cardiotoxicity in a Patient Identified as having Recieved an Anti-Neoplastic Agent

Patient Selection Criteria

Patients who have received anthracyclines within the past 10 years and are found to have unexplained deterioration in cardiac systolic function will be considered for the study. Criteria for inclusion in the study include patients with a normal baseline assessment of left ventricular systolic function prior to the administration of anthracycline, and decline in cardiac function of at least 10% points on echocardiography, nuclear or angiographic ventriculography or cMRI without known interval of mycocardial infarction or ischemia.

Patients are excluded from study if patients have history of significant obstructive valvular heart disease, obstructive hypertrophic cardiomyopathy or active myocarditis, unstable angina symptoms, MI, stroke or cardiac surgery (including percutaneous intervention) within three months prior to baseline, known hypersensitivity to xanthine oxidase inhibitors, serum creatinine greater then 3.5 mg/dl, received another investigational drug or device within 30 days prior to screening, current substance' abuse, significant neutropenia, significant hepatic renal or other disease that may limit survival to one year or less, symptomatic hyperuricemia which currently requires treatment with allopurinol and cannot be effectively treated with uricosurics or cholchicine. In addition, a patient who is unable to have LVEF monitored with cMRI imaging, is likely to receive cardiac transplantation within one year, is female and pregnant, nursing or of childbearing potential not practicing effective contraceptive methods, and/or is considered high risk for non-compliance will be excluded.

Therapy with oxypurinol is initiated after a 2-week screening and run-in period to confirm stability of symptoms, background treatment and to obtain the required baseline assessments.

Patients are randomized to receive oxypurinol at a daily dose of 600 mg or identical appearing placebo (6 capsules). Patients with reduced renal function at baseline, i.e. serum creatinine above 3 mg/dL but less than 3.5 mg/dL will receive only 3 capsules of study medication daily throughout the trial. Patients who experience deterioration of renal function during the study will have their dosage reduced to 3 capsules per day, until renal function improves and serum creatinine returns to below 3 mg/dL.

Evaluation

Following therapy, patients are assessed in the clinic at 1, 3 and 6 months using the same evaluation criteria discussed above.

### Example 3

### A Method for Modulating Anti-Neoplastic Agent-Induced Cardiotoxicity in a Patient

Patient Selection Criteria

Patients at increased risk for developing a cardiac event are patients with cardiac dysfunction prior to receiving DOX, hypertension, elderly, women, young children, and prior or concurrent radiation therapy.

DOX is administered intravenously by slow IV push. The doses in those at increased risk for a cardiac event are generally lower cumulative doses to a maximum of 400 mg/m². As a single agent, 60 to 75 mg/m² IV as a single dose is administered every 21 days, or 30 mg/m² IV for 3 days every 4 weeks. When used in combination therapy, 40-50 mg/m² as a single IV injection is administered every 21 to 28 days. The doses will be adjusted if the patient has inadequate bone marrow reserve caused by old age, has had prior therapy, or neoplastic marrow infiltration. Dose modifications are recommended for the following serum bilirubin levels: for 1.0 to 2.3 mg, reduce the dose by 50%; for 3.1 to 5.0 mg reduce the dose by 75%. Additionally, the dose may be modified as determined by the treating oncologists depending on the type of tumor and the disease stage.

Oxpurinol is administered intravenously 10-15 minutes prior to receiving DOX. The IV dosing is a 400 mg dose administered by controlled infusion pump over 15 minutes. The volume of infusate is 100 ml and the infusion rate is at 6.67 cc/min or 400 cc/hour. After the first cycle, oxypurinol may be administered orally. Patients are randomized to receive DOX+oxypurinol at a daily dose of 600 mg oxypurinol or identical appearing placebo (6 capsules/100 mg/capsule). Patients with reduced renal function at baseline, i.e. serum creatinine above 3 mg/dL but less than 3.5 mg/dL will receive only 3 capsules of study medication daily throughout the trial. Patients who experience deterioration of renal function during the study will have their dosage reduced to 3 capsules per day, until renal function improves and serum creatinine returns to below 3 mg/dL.

Evaluation

Following therapy, patients are assessed in the clinic at 1, 3 and 6 months using the same evaluation criteria discussed above.

### Example 4

### A Method for Co-Administration of a Anti-Neoplastic Agent and Oxypurinol

DOX is administered as a single agent, 60 to 75 mg/m² IV as a single dose every 21 days, or 30 mg/m² IV for 3 days every 4 weeks. When used in combination therapy, 40-50 mg/m² as a single IV injection is administered every 21 to 28 days. The doses will be adjusted if the patient has inadequate bone marrow reserve caused by old age, has had prior therapy, or neoplastic marrow infiltration. Dose modifications are recommended for the following serum bilirubin levels: for 1.0 to 2.3 mg, reduce the dose by 50%; for 3.1 to 5.0 mg reduce the dose by 75%. Additionally, the dose may be modified as determined by the treating oncologists depending on the type of tumor and the disease stage.

Oxpurinol is administered intravenously 10-15 minutes prior to receiving DOX. The IV dosing is a 400 mg dose administered by controlled infusion pump over 15 minutes. The volume of infusate is 100 ml and the infusion rate is at 6.67 cc/min or 400 cc/hour. After the first cycle, oxypurinol may be administered orally. Patients randomized to receive DOX+oxypurinol are given identical regimen as described above, i.e., 600 mg/day oxypurinol orally, or identical appearing placebo (6 capsules) concurrently with administration of anthracylines.

Patients with reduced renal function at baseline (serum creatinine above 3 mg/dL but less than 3.5 mg/dL) will receive only 3 capsules of study medication daily through out trial. Patients, who experience a deterioration of renal function during the study, must have their dosage of study medication reduced to 3 capsules per day, until such time as the renal function improves and serum creatinine returns to below 3 mg/dL.

### Example 5

### A Method for Staged Administration of an Anti-Neoplastic Agent and Oxypurinol

DOX is administered as a single agent, 60 to 75 mg/m² IV as a single dose every 21 days, or 30 mg/m² IV for 3 days every 4 weeks. When used in combination therapy, 40-50 mg/m² as a single IV injection is administered every 21 to 28 days. The doses will be adjusted if the patient has inadequate bone marrow reserve caused by old age, has had prior therapy, or neoplastic marrow infiltration. Dose modifications are recommended for the following serum bilirubin levels: for 1.0 to 2.3 mg, reduce the dose by 50%; for 3.1 to 5.0 mg reduce the dose by 75%. Additionally, the dose may be modified as determined by the treating oncologists depending on the type of tumor and the disease stage.

Oxypurinol maybe administered for three days prior to receiving the DOX and continued throughout the treatment course. Because late toxicity may occur the oxypurinol should be given for many years following completion of the chemotherapeutic regimen.

Patients randomized to receive DOX+oxypurinol are given identical regimen as described above plus 600 mg/day oxypurinol orally or identical appearing placebo (6 capsules) concurrently with administration of anthracyclines. Patients with reduced renal function at baseline (serum creatinine above 3 mg/dL but less than 3.5 mg/dL) will receive only 3 capsules of study medication daily throughout the trial. Patients, who experience a deterioration of renal function during the study, must have their dosage of study medication reduced to 3 capsules per day, until such time as the renal function improves and serum creatinine returns to below 3 mg/dL.

### Example 6

### Doxirubicin Cardiotoxicity Model

Female CD1 mice, weighing 20-22 g, were obtained from Charles River with implanted jugular catheters. Prior to induction of doxorubicin cardiotoxicity, the animals were randomly divided into 2 groups:
Group 1: Treatment with Oxypurinol (n=15), 26 mg/dl in the drinking water, start 3 days prior to doxorubicin dose and continue to sacrifice.
Group 2: Treatment with Vehicle (Saline) (n=10), start 3 days prior to doxorubicin dose and continue to sacrifice.

For induction of doxorubicin cardiotoxicity, doxorubicin, dissolved in saline, was injected by IV through the jugular catheter twice a week (Monday and Thursday) ten times at dose 4 mg/kg/day. Between doses 4 and 5 there was a two weeks hiatus to allow recovery of bone marrow suppression.

Seventeen mice were included in the vehicle Group 2. Two mice had jugular catheters that were nonoperable and excluded at the outset. Fifteen mice were included in the drug Group 1. Following 3 doses of doxorubicin there were 6 deaths in the vehicle Group 2 and only 3 deaths in the drug Group 1. Because of the unexpected high rate of death a decision was made to sacrifice the mice prior to dose 4 of doxorubicin, for histological analysis.

Seven surviving vehicle Group 2 (control) animals were sacrificed and 11 surviving drug Group 1 mice were sacrificed. The mean cellular swelling score was 2.6±0.5 and 1.8±0.4. This is highly significant p=0.004. The cellular necrosis score was not statistically significant.

Given the highly significant decrease in mortality in the oxypurinol Group 1 and the highly significant difference in the swelling score it appears highly likely that oxypurinol prevents death and cellular swelling caused by doxorubicin cardiotoxicity. The lack of difference in the necrosis scoring is probably due to time to first event bias. It is expected from these results that oxypurinol, as a xanthine oxidase inhibitor, ameliorates or prevents adverse cardiac events associated with anti-neoplastic agent, such as doxorubicin, induced cardiotoxicity.

Throughout this application, reference has been made to various publications, patents and patent applications. The teachings and disclosures of these publications, patents and patent applications in their entireties are hereby incorporated by reference into this application to more fully describe the state of the art to which the present invention pertains.

What is claimed:

1. A method for ameliorating or preventing adverse cardiac events in a patient who is at risk for, or who is suffering from, a neoplastic condition, comprising administering to the patient a biologically effective amount of an xanthine oxidase inhibitor.

2. The method of claim 1 wherein the xanthine oxidase inhibitor is oxypurinol, salts or derivatives thereof.

3. A method of ameliorating or preventing anti-neoplastic agent-induced cardiotoxicity in a patient at risk for or suffering from a neoplastic condition comprising administering:
(a) the anti-neoplastic agent, and
(b) oxypurinol, salts or derivatives thereof, in a biologically effective amount to ameliorate or prevent cardiotoxicity in said patient.

4. A method of ameliorating or preventing anti-neoplastic agent-induced cardiotoxicity comprising:
(a) identifying a patient treated with the anti-neoplastic agent; and
(b) administering oxypurinol, salts or derivatives thereof, in a biologically effective amount sufficient to ameliorate or prevent cardiotoxicity in said patient.

5. A method of ameliorating or preventing cardiotoxicity comprising:
(a) identifying a patient suffering from a neoplastic disease at risk for or suffering from an adverse cardiac event; and
(b) administering oxypurinol, salts or derivatives thereof, in a biologically effective amount sufficient to ameliorate or prevent cardiotoxicity in said patient.

6. The method of claim 5, further comprising the step of identifying whether the patient was exposed to an anti-neoplastic agent prior to performing step (b).

7. The method of claim 6, further comprising the step of administering an anti-neoplastic agent prior to or contemporaneous with performance of step (b).

8. A method for ameliorating or preventing anti-neoplastic agent-induced cardiotoxicity by identifying a patient who is suffering from a neoplastic disease or at risk for, and/or suffering from an adverse cardiac event, and administering a biologically effective amount of an xanthine oxidase inhibitor.

9. The method of claim 8 wherein the xanthine oxidase inhibitor is oxypurinol, salts or derivatives thereof.

10. A method for ameliorating or preventing anti-neoplastic agent-induced cardiotoxicity by identifying a patient treated with an anti-neoplastic agent and administering a biologically effective amount of an xanthine oxidase inhibitor sufficient to ameliorate or prevent cardiotoxicity in the patient.

11. The method of claim 10 wherein the xanthine oxidase inhibitor is oxypurinol, salts or derivatives thereof.

12. A method of ameliorating or preventing anti-neoplastic agent-induced cardiotoxicity comprising co-administering a biologically effective amount of an anti-neoplastic agent and xanthine oxidase inhibitor.

13. The method of claim 12 wherein the xanthine oxidase inhibitor is oxypurinol, salts or derivatives thereof.

14. A method for ameliorating or preventing an anti-neoplastic agent-induced cardiotoxicity by staged administration of a biologically effective amount of an xanthine oxidase inhibitor before, during, or after delivery of the anti-neoplastic agent.

15. The method of claim 14 wherein the xanthine oxidase inhibitor is oxypurinol, salts or derivatives thereof.

16. The method of claim 3, 4, 8, 10, 12 or 14 wherein the anti-neoplastic agent-induced cardiotoxicity is selected from the group consisting of cardiac failure, left ventricular failure, cardiomyopathy, myocarditis, pericarditis, myocardial infarction, arrthymia, blood pressure change, congestive heart failure, thrombosis, and electrocardiographic change.

17. The method of claim 3, 4, 6, 8, 10, 12 or 14 wherein the anti-neoplastic agent is an anthracycline, including but not limited to, doxorubicin, daunorubicin, idarubicin, detorubicin, carminomycin, epirubicin, morpholinodoxorubicin, morpholinodaunorubicin, methoxymorpholinyldoxorubicin, substitutes, derivatives, and one or more combinations thereof.

18. The method of claim 13 or 15 wherein the anti-neoplastic agent and the oxypurinol are delivered intravenously, orally, intramuscularly, or intraperitoneally.

19. The method of claim 13 or 15 wherein the oxypurinol is delivered utilizing the same method of delivery as the anti-neoplastic agent.

20. A kit for delivery of one or more doses of an anti-neoplastic agent, and one or more doses of oxypurinol, salts or derivatives thereof.
